(19) United States
(12) Patent Application Publication
(10) Pub. No.: US 2013/0303811 A1
(43) Pub. Date: Nov. 14, 2013

(54) GAS TREATMENT PROCESS

(71) Applicant: AXENS, Rueil Malmaison Cedex (FR)

(72) Inventors: Eric ROISIN, Rueil Malmaison (FR); Nicolas LAMBERT, Issy-Les-Moulineaux (FR); Yann LOONIS, Paris (FR)

(21) Appl. No.: 13/887,442

(22) Filed: May 6, 2013

(30) Foreign Application Priority Data
May 10, 2012 (FR) 1201362

(51) Int. Cl. C07C 5/02; C07C 7/148

(52) U.S. Cl. CPC C07C 5/02; C07C 7/14866
USPC 585/251

(57) ABSTRACT

The present invention relates to a process for treating a gas containing from 10 ppm by volume to 0.5 volume % of at least one of the compounds COS and CS₂ and from 30 ppm by volume to 5 volume % of unsaturated hydrocarbon compounds, the said process comprising the following stages:

(a) a hydrogenation of the unsaturated organic compounds to paraffins is carried out by contacting the said gas with a hydrogenation catalyst in the presence of hydrogen,
(b) a catalytic hydrolysis of COS and/or CS₂ present in the gaseous effluent from stage a) is carried out in the presence of water and a hydrolysis catalyst.

This application claims the benefit of priority to French Patent Application No. 1201362, filed on May 10, 2012, and is incorporated herein by reference in its entirety. The entire disclosures of all applications, patents and publications, cited herein are incorporated by reference herein in their entirety.

### FIELD OF THE INVENTION

The invention relates to a process for treating gas that contains in particular sulphur-containing compounds as impurities to be removed. In particular the process applies to the treatment of gaseous effluent discharged from furnaces of a carbon black production unit.

### PRIOR ART

Various industrial processes exist that emit gaseous effluents containing sulphur. These effluents or residual gases have to be treated before being discharged into the atmosphere in order to reduce the amount of these polluting sulphur-containing compounds to acceptable levels, so as to comply with the increasingly stringent environmental standards.

The sulphur found in industrial residual gases, for example in the gases emitted by coal, petcoke (petroleum coke) or also biomass gasification units, or in the gases emitted by calcination furnaces of carbon black production units, is generally in the form of sulphur dioxide, hydrogen sulphide and carbon sulphides such as carbon disulphide (CS₂) and carbonyl sulphide (COS). The carbon sulphides are relatively inert compounds and are therefore difficult to remove efficiently from the gaseous effluent.

Various methods are known in the prior art for removing sulphur. These methods are generally based on the principle of forming hydrogen sulphide (H₂S), which is a reactive compound and can easily be removed.

Thus, for example, the document U.S. Pat. No. 5,466,427 discloses a process for treating a residual gas containing sulphur, particularly in the form of carbon sulphides, which consists in contacting the said gas with a catalyst so as to effect a hydrolysis of carbon disulphide (CS₂) and carbonyl sulphide (COS) to form hydrogen sulphide. The catalyst used in this document comprises:
0.5-5 wt. % of cobalt oxide and/or nickel oxide;
1.5-15 wt. % of molybdenum trioxide;
70-98 wt. % of titanium oxide.

As noted by the applicant, the prior art process operates satisfactorily provided that the gas to be treated contains a relatively small amount of unsaturated hydrocarbon products (in particular alkyne and diene type compounds), that is to say in a maximum concentration of 50 ppm, or even 30 ppm by volume.

### DESCRIPTION

An object of the invention is to provide a process for treating a gas capable of converting the sulphur-containing compounds present in particular in the form of CS₂ and/or COS that is operational even if the feedstock to be treated contains unsaturated hydrocarbon compounds, for example in a concentration greater than 30 ppm by volume.

To this end the process according to the invention comprises the following stages:
a) a hydrogenation of the unsaturated hydrocarbon compounds to paraffins is carried out by contacting the said gas with a hydrogenation catalyst in the presence of hydrogen at a temperature between 100 and 400° C., so as to provide a gaseous effluent depleted in unsaturated hydrocarbon compounds, the hydrogenation catalyst comprising at least one metal selected from palladium, platinum, nickel and cobalt deposited on a porous support,
b) a catalytic hydrolysis of COS and/or CS₂ present in the gaseous effluent from stage a) is carried out in the presence of water so as to provide a gaseous effluent rich in H₂S, by contacting the said gaseous effluent from stage a) with a hydrolysis catalyst, the hydrolysis catalyst comprising alumina or titanium oxide.

It has been found that the stage a) involving hydrogenation of the feedstock, prevents the progressive deactivation, or even an obstruction of the hydrolysis catalyst of stage b), particularly as a result of the formation of polymerisation gums on the surface of the catalysts. In fact, thanks to the pre-treatment of the feedstock in stage a) the unsaturated hydrocarbon compounds are converted by hydrogenation into compounds that are not liable to polymerise and therefore poison or lead to coking or obstruction of the pores of the hydrolysis catalyst in the subsequent stage b), the result being an improved efficiency of the treatment process compared to the prior art.

In the context of the invention the term “unsaturated hydrocarbon compounds” covers in particular alkene and alkyne type compounds and also diene type polyunsaturated compounds.

According to one embodiment the stages a) and b) are carried out in the same reactor, in which two catalyst beds, namely a hydrogenation catalyst and a hydrolysis catalyst, are arranged in succession. The catalyst beds are disposed with respect to one another in the reactor in such a way that the feedstock to be treated comes into contact with the hydrogenation catalyst bed before the hydrolysis catalyst bed.

According to another alternative embodiment, the process employs two specific reactors (i.e. a hydrogenation reactor and a hydrolysis reactor), in which the hydrogenation reactor is installed downstream of the hydrolysis reactor.

According to a preferred embodiment the process according to the invention comprises a treatment stage of the gas leaving the hydrolysis stage, which consists for example in trapping the H₂S that is formed or converting the H₂S into elementary sulphur.

According to an advantageous embodiment a liquid/gas separation stage of the gas to be treated may be performed before carrying out the hydrogenation stage.

Likewise it is possible to carry out a liquid/gas separation of the gas leaving the stage b) before passing it to an H₂S treatment unit.

The Gaseous Feedstock to be Treated

The gas that can be treated by the process according to the invention may be obtained from coal or petcoke or even biomass gasification units or from calcination furnaces of carbon black production units. Typically the gas to be treated may contain COS and/or CS₂ in an amount between 10 ppm by volume and 0.5 volume %. The gas thus generally includes COS in an amount that is most often between 10 ppm by volume and 0.3 volume %, CS₂ in an amount between 10 ppm by volume and 0.3 volume % and possibly HCN in an amount between 20 ppm by volume and 0.2 volume %. The gas may also contain hydrogen, CO, SO₂, CO₂, H₂S and water.

The gas generally contains unsaturated hydrocarbons in an amount between 30 ppm by volume and 5 volume %, preferably between 0.05 and 3 volume %. Generally the unsaturated hydrocarbon compounds basically include short-chain, typically C2, C3 or C4, hydrocarbon products of the group of alkenes, alkynes and polyunsaturated compounds, such as for example ethylene, acetylene and butadiene.

The Hydrogenation Stage (Stage a):

Within the scope of the invention the hydrogenation may be selective, that is to say it involves only alkynes and diene type polyunsaturated compounds but not mono-olefins. Nevertheless, even though this is not generally necessary, there is no disadvantage in carrying out a total hydrogenation, that is to say hydrogenating all the unsaturated compounds, including mono-olefins, into paraffins.

The hydrogenation catalyst employed in the stage a) comprises a metal chosen from platinum, palladium, nickel and cobalt individually or as a mixture, and deposited on a porous support.

According to a first variant of the process according to the invention, the hydrogenation catalyst comprises platinum, and the content of platinum, expressed as metal, is normally between 0.02 wt. % and 4 wt. % with respect to the total weight of the catalyst. Preferably the content of platinum is between 0.05 and 3 wt. %, more preferably between 0.1 wt. % and 2.5 wt. % with respect to the total weight of the catalyst.

According to a second variant of the process according to the invention, the hydrogenation catalyst comprises palladium and the content of palladium, expressed as metal, is between 0.05 wt. % and 5 wt. % with respect to the total weight of the catalyst. Preferably the content of palladium is between 0.05 and 3 wt. %, more preferably between 0.1 wt. % and 1 wt. % with respect to the total weight of the catalyst.

According to a third variant of the process according to the invention, the hydrogenation catalyst comprises nickel and the content of nickel is generally between 0.5 wt. % and 15 wt. % of nickel oxide with respect to the total weight of the catalyst. Preferably the content of nickel oxide is between 4 wt. % and 12 wt. %, more preferably between 6 wt. % and 10 wt. % with respect to the total weight of the catalyst.

According to a fourth variant of the process according to the invention, the hydrogenation catalyst comprises cobalt and the content of cobalt is generally between 0.5 wt. % and 15 wt. % of cobalt oxide with respect to the total weight of the catalyst. Preferably the content of cobalt oxide is between 1 wt. % and 10 wt. %, more preferably between 2 wt. % and 4 wt. % with respect to the total weight of the catalyst.

According to a particular embodiment the hydrogenation catalyst contains either platinum, or palladium, or nickel, or cobalt, and may also include molybdenum. In this case the molybdenum content, expressed as molybdenum oxide, of the said catalyst is between 1 wt. % and 20 wt. % with respect to the total weight of the catalyst, preferably between 6 wt. % and 18 wt. %, and more preferably between 8 wt. % and 15 wt. %.

The catalyst of stage a) is a catalyst that also comprises a porous support on which are deposited the metal(s) or precursor(s) of the metals or oxides active in hydrogenation. The support may be chosen from aluminas, silicas, titanium oxide, silicon carbide or their mixtures.

The porous support is preferably chosen from alumina, nickel or cobalt aluminate, silica, silica-aluminas, silicon carbon, titanium oxide or their mixtures. Pure alumina or titanium oxide is preferably used.

According to a very preferred variant, the support consists of cubic gamma alumina or delta alumina.

More preferably the hydrogenation catalyst employed in stage a) comprises palladium. According to another preferred embodiment it comprises nickel and molybdenum.

The catalyst according to the invention may be prepared by any means known to the person skilled in the art, and in particular by impregnating metallic elements on the selected support. This impregnation may for example be realised according to the method known to the person skilled in the art by the term dry impregnation, in which exactly the amount of desired elements is introduced in the form of salts that are soluble in the chosen solvent, for example demineralised water, so as to fill as completely as possible the pores of the support. The support thus filled by the solution is preferably dry. The preferred support is alumina or titanium oxide, which may be prepared from any type of precursors and moulding tools known to the person skilled in the art.

After introducing the precursors of the metallic elements, and possibly moulding the catalyst, the latter is subjected to a thermal treatment comprising a drying stage followed by a calcination. The drying is generally carried out in air between 20° C. and 200° C., preferably between 40° C. and 180° C. The calcination is generally performed in air or in dilute oxygen, and the treatment temperature is generally between 200° C. and 550° C., preferably between 300° C. and 500° C.

The Hydrolysis Stage (Stage b):

The hydrogenation catalyst employed in the stage b) is a catalyst that comprises alumina or titanium oxide, preferably titanium oxide. The catalyst according to the invention may also include at least 1 wt. %, preferably between 0.5 wt. % and 10 wt. %, and more preferably between 1 wt. % and 5 wt. %, of at least one sulphate or silicate of an alkali or alkaline earth metal or of a rare earth.

The said alkali metal is preferably chosen from lithium, sodium, potassium and, more preferably, from sodium or potassium. The said alkaline earth metal is preferably chosen from calcium, barium, strontium and magnesium. The rare earth is preferably chosen from lanthanum, cerium, praseodymium or neodymium. Most preferably the rare earth is lanthanum.

More preferably the catalyst contains a silicate or a sulphate of sodium, potassium, calcium or barium. Most preferably it comprises calcium or barium sulphate and, still more preferably, calcium sulphate.

According to a particular embodiment the hydrolysis catalyst also comprises a metal chosen from nickel, cobalt, molybdenum and tungsten.

According to another variant of the process according to the invention, the hydrolysis catalyst used in stage b) comprises 60 wt. % to 99.8 wt. % of titanium oxide or alumina with respect to the weight of the catalyst, and also a metal chosen from nickel, cobalt, molybdenum and tungsten, individually or as a mixture.

According to a preferred variant the hydrolysis catalyst comprises:
titanium oxide or alumina in an amount between 60% and 99.8 wt. % with respect to the weight of the catalyst,
at least one metal chosen from nickel, cobalt, molybdenum and tungsten in an amount, expressed as oxide, between 0.2 wt. % and 40 wt. %, preferably between 1 wt. % and 25 wt. % with respect to the weight of the catalyst.

According to an even more preferred variant the said hydrolysis catalyst comprises nickel and molybdenum or cobalt and molybdenum supported on a titanium oxide support.

According to another very preferred variant, the hydrolysis catalyst used in stage b) comprises:
nickel in an amount, expressed as wt. % of oxide, between 0.5 and 15 wt. %, preferably between 1% and 8 wt. % with respect to the weight of the catalyst;
cobalt in an amount, expressed in wt. % of oxide, between 0 and 10 wt. %, preferably between 0.5 and 5 wt. %, with respect to the weight of the catalyst;
molybdenum or tungsten in an amount, expressed in wt. % of oxide, between 1% and 18 wt. %, preferably between 5 wt. % and 15 wt. % with respect to the weight of the catalyst;
titanium oxide in an amount, expressed in wt. % of oxide, between 60% and 98.5% with respect to the weight of the catalyst, preferably between 70% and 98%.

Preferably the titanium oxide has a rutile or anatase crystallographic structure.

The catalyst may be prepared by any technical means known to the person skilled in the art, and in particular by impregnating precursors of nickel, cobalt, molybdenum, sulphate or silicate of an alkali metal or alkaline earth metal or of a rare earth on the previously moulded support.

This impregnation may be realised for example according to the method known to the person skilled in the art by the term “dry impregnation”, in which exactly the amount of desired elements is introduced in the form of salts soluble in the chosen solvent, for example demineralised water, so as to fill the pores of the support as completely as possible.

The impregnated support may then be dried, preferably at a temperature between 20° C. and 200° C., more preferably between 40° C. and 180° C. A calcination is then generally carried out in air or in diluted oxygen, and the calcination temperature is generally between 200° C. and 550° C., preferably between 300° C. and 500° C.

### BRIEF DESCRIPTION OF THE FIGURES

These aspects as well as other aspects of the invention will become clear in the following detailed description of particular embodiments of the invention, and with reference to the accompanying drawings, in which:

FIG. 1 shows a first embodiment of the process according to the invention.

FIG. 2 shows a second embodiment of the process according to the invention.

FIG. 3 shows a third embodiment of the process according to the invention.

The figures are not drawn to scale. Generally the same elements are denoted by identical reference numerals in the figures.

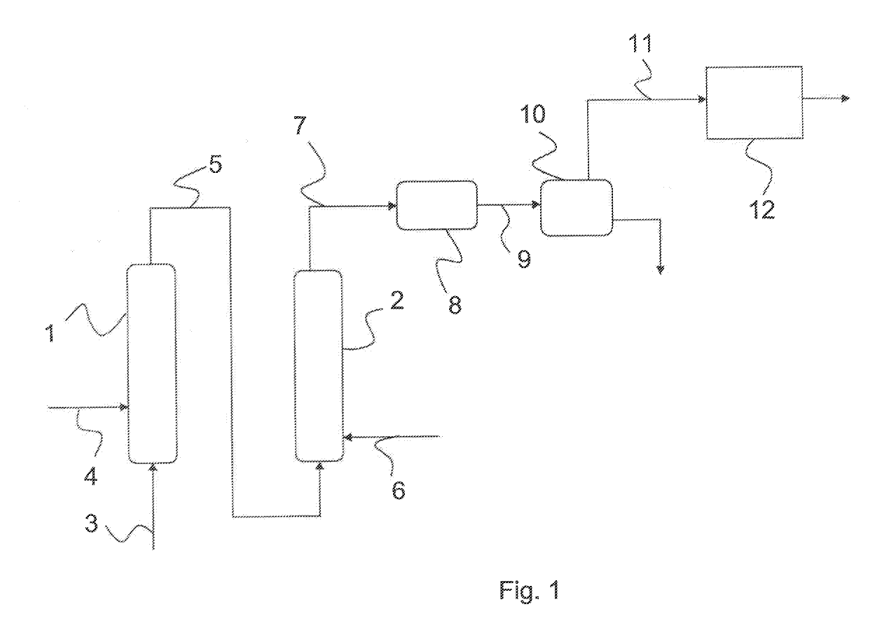

With reference to FIG. 1, the process involves a first reactor 1 which contains a catalyst for hydrogenating unsaturated compounds, preferably a selective hydrogenation catalyst. Any previously described hydrogenation catalyst may be used in this embodiment.

As shown in FIG. 1, the gaseous feedstock to be treated is introduced into the reactor 1 via the line 3, while an additional amount of hydrogen, supplementing the hydrogen initially present in the gas to be treated, may if necessary be introduced into the reactor 1 via the line 4.

The total amount of hydrogen present in the gas to be treated and possibly added hydrogen is such that the molar ratio between hydrogen and the unsaturated hydrocarbon compounds to be hydrogenated is greater than the stoichiometric amount and is preferably between 1 and 3000 moles per mole and preferably between 300 and 2000 moles per mole.

The hydrogenation stage is generally carried out at a pressure between 0.1 and 5 MPa, preferably between 0.5 and 3 MPa, at a temperature between 100 and 400° C., preferably between 150° C. and 250° C., and a catalyst volume with respect to the amount of gas to be treated with 1 m³ of catalyst for 1000 to 4000 Nm³/h of gas to be treated, i.e. a HSV between 1000 and 4000 h⁻¹.

With reference to FIG. 1, the gaseous effluent leaving the hydrogenation reactor is then passed to the hydrolysis reactor 2 via the line 5, in which the conversion of the sulphur-containing compounds COS and CS₂ into H₂S is carried out on a specific catalyst in the presence of water.

If the water content in the feedstock is not sufficient, then additional water may be introduced via the line 6, so as to carry out the hydrolysis with an excess of water with respect to the hydrolysable molecules (COS, CS₂, HCN).

Any afore described hydrolysis catalyst may be used in this embodiment.

The reactions that take place during this stage may be represented by the following conversions:

COS+H₂O→CO₂+H₂S

CS₂+2H₂O→2H₂S+CO₂

The hydrolysis stage is typically carried out at a pressure between 0.1 and 5 MPa, preferably between 0.5 and 3 MPa, at a temperature between 100 and 400° C., preferably between 150° C. and 250° C., and a catalyst volume with respect to the amount of gas to be treated of 1 m³ of catalyst for 1000 to 4000 Nm³/h of gas to be treated, i.e. a HSV between 1000 and 4000 h⁻¹.

The hydrolysis is carried out in the presence of an excess of water with respect to the molecules to be hydrolysed. Preferably the reaction is carried out with a molar ratio of water to hydrolysable products of between 5 and 1000 moles per mole, and more preferably between 10 and 500 moles per mole.

The gaseous effluent treated in the hydrolysis reactor 2 is then extracted and led via the line 7 to a heat exchanger 8, for example a cooling tower, so as to cool the treated gas. The treated and cooled gas is transferred by the line 9 to a liquid/gas separator 10. The liquid condensation water is recovered at the bottom of the separator 10, while the gas depleted in H₂O and containing H₂S is led via the line 11 to a treatment unit 12, which may for example be a unit for trapping H₂S or a unit for converting H₂S, which for example oxidises H₂S to form elementary sulphur:

2H₂S+SO₂→3S+2H₂O

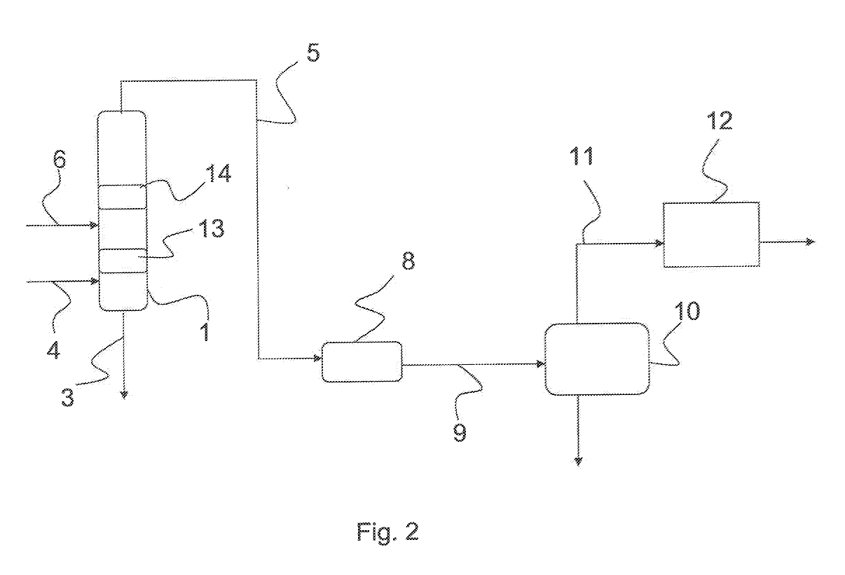

According to the invention the two hydrogenation and hydrolysis reactions of the feedstock to be treated may be carried out in the same reactor comprising a first hydrogenation catalyst bed and a second hydrolysis catalyst bed, the beds being arranged with respect to one another in the reactor so that the gaseous feedstock to be treated comes into contact with the hydrogenation catalyst bed before the hydrolysis catalyst bed, as shown in FIG. 2.

With reference to FIG. 2, the second embodiment employs a single reactor 1 in which the catalytic hydrogenation and hydrolysis reactions are carried out. To this end, the reactor comprises two catalyst beds 13 and 14, respectively for hydrogenation and hydrolysis. These two beds may be separated from one another by an internal space or instead may be consecutive without any intermediate space. The catalyst beds 13 and 14 are arranged in the reactor 1 in such a way that the feedstock to be treated first comes into contact with the catalytic hydrogenation bed 13 and then the catalytic hydrolysis bed.

In order to carry out the hydrogenation of the gaseous feedstock which is introduced via the line 3, an additional supply of hydrogen may possibly be implemented by means of the line 4 situated upstream of the catalyst bed 13. If necessary an internal space separates the catalytic beds 13 and 14, so as to arrange in this space an injection point for adding via the line 6 extra water necessary for the hydrolysis reaction.

The operating conditions used for the two catalytic reactions and described with reference to FIG. 1 are applicable in this second embodiment. Any hydrogenation or hydrolysis catalyst described hereinbefore may also be used in this embodiment.

In a similar manner to the embodiment of FIG. 1, the gaseous effluent from the catalytic hydrolysis is removed from the reactor 1 and conveyed via the line 5 to a heat exchanger 8 and then to a separating flask 10 via the line 9. The condensation water is extracted from the bottom of the separating flask 10 and a gas loaded with H₂S is discharged from the head of the flask and transferred to a unit 12 for trapping or converting H₂S.

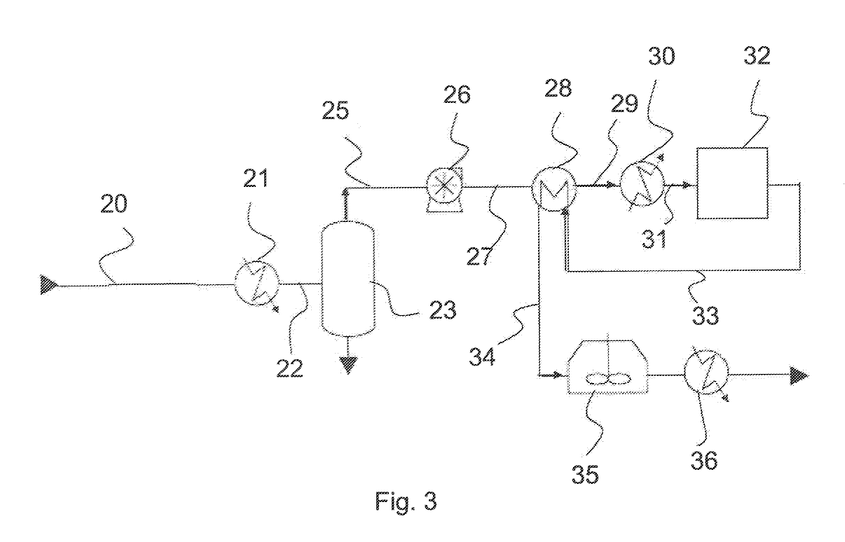

The third embodiment of the process according to the invention is represented in FIG. 3 and differs basically from the embodiments of FIGS. 1 and 2 in that it comprises a prior gas/liquid separation stage carried out on the gas to be treated. It is in fact important to remove basically excess water and/or possibly liquid organic compounds dissolved or not in the gaseous phase so as to reduce the feedstock volume to be treated, while maintaining an excess of water for carrying out the hydrolysis reaction.

In this case, as shown in FIG. 3, the gas to be treated, which is generally hot, is passed via the line 20 to a heat exchanger 21, where it is cooled, and is then passed via the line 22 to a separating flask 23. In the flask 23 two phases are separated, namely a gaseous phase at the head of the flask and a liquid phase at the bottom, which contains the water of the feedstock. The gas separated from this liquid and possibly from part of the dissolved water leaving the separating flask 23 is passed via the line 25 to a compressor 26, where it is compressed.

As shown in FIG. 3, the compressed gas possibly undergoes a heating stage via the optional heat exchanger 28, which if present is supplied with a hot fluid. This hot fluid is, preferably and according to the example of FIG. 3, the hot gaseous effluent leaving the hydrogenation and hydrolysis reaction zone 32. The previously heated compressed gas may optionally be raised to the operating temperature by means of an optional heating unit 30, for example a heat exchanger, before being introduced into the reaction zone 32, where the hydrogenation and hydrolysis reactions are carried out according to the process of the invention. The hot effluent leaving the reaction zone 32 is discharged via the line 33 and introduced into the heat exchanger 28 in order to heat the gas to be treated. After having been cooled by contact with the gas to be treated, the treated gas rich in H₂S may possibly be passed via the line 34 to a second optional condenser 35, for example a cooling tower, and/or possibly to an also optional heat exchanger 36. This cooling enables the temperature of the flow to be adjusted to a temperature compatible with possible upstream treatments. Thus, the cooled flow may in particular then be treated in a unit (not shown) for trapping H₂S or converting H₂S into elementary sulphur.

### EXAMPLE

The Examples 1 (comparative), 2 and 3 (according to the invention) relate to the efficiency of the conversion of the sulphur compounds in the form of H₂S of a typical feedstock A whose composition is shown in Table 1. The feedstock A corresponds to a gaseous effluent leaving a carbon black production unit and contains sulphur and nitrogen compounds (COS, CS₂ and HCN) and acetylene. Example 1 is carried out according to the process described in the U.S. Pat. No. 5,466,427.

The Examples 2 and 3 are carried out according to the process of the invention, comprising at least one first stage a) for hydrogenating the unsaturated hydrocarbon compounds present in the feedstock A, followed by a stage b) for the catalytic hydrolysis of the sulphur and nitrogen compounds (COS and/or CS₂ and HCN) present in the effluent leaving the stage a).

TABLE 1
Composition of the feedstock A

| Composition of the | Content |
|---|---|
| feedstock A | (vol. %) |
| CS₂ | 0.08 |
| HCN | 0.05 |
| COS | 0.013 |
| SO₂ | 0.007 |
| H₂S | 0.15 |
| CH₄ | 0.3 |
| C₂H₂ | 0.3 |
| CO | 8 |
| H₂ | 9 |
| CO₂ | 2 |
| H₂O | 45 |
| O₂ | 0.1 |
| N₂ | 35 |

The gaseous feedstock to be treated thus contains a not negligible amount of acetylene of up to 0.3 vol. %.

### Example 1

### Comparative

The feedstock A is passed directly together with water to a reactor for the hydrolysis of the compounds COS and CS₂ (reactor dimensions: diameter 2 cm, height 10 cm) containing a catalyst prepared by impregnating a support based on titanium dioxide (TiO₂) in the form of extruded cylindrical pellets of diameter 3.2 mm and average length 6.7 mm, on which is deposited nickel and molybdenum. The catalyst has the following composition: 2.5 wt. % of nickel oxide (NiO), 9.0 wt. % of molybdenum trioxide (MoO₃) and 88.5% wt. % of titanium dioxide. The weight % are expressed with respect to the total weight of the catalyst.

The operating conditions for the catalytic hydrolysis reaction are as follows:
temperature (° C.): 220
pressure (MPa): 0.2
HSV (h⁻¹): 3600
total content of water: 45 vol. %

The composition of the effluent leaving the reactor is analysed (by gaseous phase chromatography). The results obtained after 48 hours' operation are shown in Table 2.

TABLE 2
Composition of the gaseous effluent leaving the hydrolysis stage

| Composition | |
|---|---|
| of the effluent | |
| leaving the | Content |
| reactor | (vol. %) |
| CS₂ | 0.07 |
| HCN | 0.03 |
| COS | 0.005 |
| SO₂ | 0.005 |
| H₂S | 0.16 |
| CH₄ | 0.3 |
| C₂H₂ | 0.3 |
| CO | 8 |
| H₂ | 9 |
| CO₂ | 2 |
| H₂O | 45 |
| O₂ | Not determined |
| N₂ | 35 |
| NH₃ | 0.02 |

After 48 hours' operation a reduction of the content of CS₂ of the order of barely 12% is observed. In contrast, a reduction of the content of COS of 61% is found. Thus, the hydrolysis catalyst exhibits a low hydrolysis activity for carbon disulphide when the reaction is carried out with a gas containing unsaturated organic compounds (in the present case acetylene).

### Example 2

### According to the Invention

The same feedstock A whose composition was given in Table 1 is first of all passed to a first hydrogenation reactor according to stage a) of the invention. The hydrogenation catalyst used in stage a) consists of 0.28 wt. % of Pd on a support consisting of agglomerated gamma alumina in the form of spheres of diameter 1.7 mm. The stage a) is carried out under the following operating conditions:
temperature (° C.): 220
pressure (MPa): 0.2
HSV (h⁻¹): 3200
content of H₂ in the feedstock: 9 vol. %, no extra hydrogen is added.

The effluent leaving stage a) is analysed after 48 hours' operation according to the method described in Example 1 and has the composition given in Table 3.

TABLE 3
composition of the effluent from the hydrogenation stage

| Effluent leaving | Content |
|---|---|
| stage a) | (vol. %) |
| CS₂ | 0.07 |
| HCN | 0.005 |
| COS | 0.008 |
| SO₂ | 0.001 |
| H₂S | 0.18 |
| CH₄ | 0.3 |
| C₂H₂ | <0.01 |
| C₂H₆ | 0.3 |
| CO | 7.8 |
| H₂ | 9 |
| CO₂ | 2.1 |
| H₂O | 45 |
| O₂ | <0.001 |
| N₂ | 35 |

Thus, the hydrogenation stage has enabled the concentration of acetylene to be considerably reduced, with the corresponding formation of ethane.

The effluent leaving stage a) is then passed to a second reactor according to stage b) of the invention. The catalyst used in stage b) consists of 91 wt. % of TiO₂ and 9 wt. % of CaSO₄. The stage b) is carried out under the following operating conditions:
temperature (° C.): 220
pressure (MPa): 0.2
HSV (h⁻¹): 2000
content of water in the feedstock: 45 vol. % (no extra water is added)
   The composition of the effluent leaving stage b) is given in Table 4.

TABLE 4
Composition of the effluent from the hydrolysis stage

| Effluent leaving | Content |
|---|---|
| stage b) | (vol. %) |
| CS₂ | 0.01 |
| HCN | <0.001 |
| COS | 0.005 |
| SO₂ | 0.001 |
| H₂S | 0.30 |
| CH₄ | 0.3 |
| C₂H₂ | <0.01 |
| C₂H₆ | 0.3 |
| CO | 7.8 |
| H₂ | 9 |
| CO₂ | 2.1 |
| H₂O | 45 |
| O₂ | <0.001 |
| N₂ | 35 |

It is found that the hydrogenation stage carried out on the residual gas has a positive effect on the hydrolysis yield of the compound CS₂. Thus, a reduction of the CS₂ content of the order of 86% is obtained. It may therefore be concluded that a pre-treatment of the gas in order to reduce the concentration of unsaturated organic compounds enables a better hydrolysis activity of carbon disulphide to be maintained.

### Example 3

### According to the Invention

The feedstock A described in Table 1 is first of all passed to a first reactor according to stage a) of the invention. The hydrogenation catalyst used in stage a) consists of 0.28 wt. % of Pd on a support consisting of agglomerated gamma alumina in the form of spheres of diameter 1.7 mm. The stage a) is carried out under the following operating conditions:
temperature (° C.): 220
pressure (MPa): 0.2
HSV (h⁻¹): 3200
content of H₂ in the feedstock: 9 vol. %, therefore no extra hydrogen is added.

The composition of the effluent leaving stage a) is analysed after 48 hours' operation according to the method described in Example 1. The composition of the effluent leaving stage a) is given in Table 5.

TABLE 5
Composition of the effluent from the hydrogenation stage

| Effluent leaving | Content |
|---|---|
| stage a) | (vol. %) |
| CS₂ | 0.07 |
| HCN | 0.005 |
| COS | 0.008 |
| SO₂ | 0.001 |
| H₂S | 0.18 |
| CH₄ | 0.3 |
| C₂H₂ | <0.01 |
| C₂H₆ | 0.3 |
| CO | 7.8 |
| H₂ | 9 |
| CO₂ | 2.1 |
| H₂O | 45 |
| O₂ | <0.001 |
| N₂ | 35 |

The prior hydrogenation treatment of the feedstock gas thus enables acetylene to be converted into ethane and consequently enables the content of acetylene to be reduced to a value of less than 0.01 vol. %.

The effluent leaving stage a) is then passed to and treated in a second reactor according to stage b) of the invention. The catalyst used in stage b) is that described in Example 1. The catalyst has the following composition (expressed in terms of the total weight of the catalyst): 2.5 wt. % of nickel oxide (NiO), 9.0 wt. % of molybdenum trioxide (MoO₃) and 88.5 wt. % of titanium dioxide.

The operating conditions of this stage b) are as follows:
temperature (° C.): 220
pressure (MPa): 0.2
HSV (h⁻¹): 2000
water content in the feedstock: 45 vol. %, and accordingly no extra water is added

The composition of the effluent leaving stage b) is analysed according to the method described in Example 1. The results are shown in Table 6.

TABLE 6
Composition of the effluent from the hydrolysis stage

| Effluent leaving | Content |
|---|---|
| stage b) | (vol. %) |
| CS₂ | 0.01 |
| HCN | <0.001 |
| COS | 0.005 |
| SO₂ | 0.001 |
| H₂S | 0.30 |
| CH₄ | 0.3 |
| C₂H₂ | <0.01 |
| C₂H₆ | 0.3 |
| CO | 4.6 |
| H₂ | 12.2 |
| CO₂ | 5.3 |
| H₂O | 42 |
| O₂ | <0.001 |
| N₂ | 35 |

The analyses show that the prior hydrogenation stage intended to saturate the unsaturated organic compounds enables the catalytic performance of the catalyst to be maintained, in particular the hydrolysis activity with regard to carbon sulphides.

Here too, a reduction in the content of CS₂ of the order of 87% and of COS of the order of 61% is obtained.

1. Process for treating a gas containing from 10 ppm by volume to 0.5 volume % of at least one of the compounds COS and CS₂ and from 30 ppm by volume to 5 volume % of unsaturated hydrocarbon compounds, the said process comprising the following stages:
a) a hydrogenation (1) of the unsaturated hydrocarbon compounds to paraffins is carried out by contacting the said gas with a hydrogenation catalyst in the presence of hydrogen at a temperature between 100 and 400° C., so as to provide a gaseous effluent depleted in unsaturated hydrocarbon compounds, the hydrogenation catalyst comprising at least one metal selected from palladium, platinum, nickel and cobalt deposited on a porous support,
b) a catalytic hydrolysis (2) of COS and/or CS₂ present in the gaseous effluent from stage a) is carried out in the presence of water so as to provide a gaseous effluent rich in H₂S by contacting the said gaseous effluent from stage a) with a hydrolysis catalyst, the hydrolysis catalyst comprising alumina or titanium oxide.

2. Process according to claim 1, in which the hydrogenation catalyst contains platinum, expressed as metal, in an amount between 0.02 wt. % and 4 wt. % with respect to the weight of the catalyst.

3. Process according to claim 1, in which the hydrogenation catalyst contains palladium, expressed as metal, in an amount between 0.05 wt. % and 5 wt. % with respect to the weight of the catalyst.

4. Process according to claim 1, in which the hydrogenation catalyst contains nickel in an amount, expressed as oxide, between 0.5 wt. % and 15 wt. % with respect to the weight of the catalyst.

5. Process according to claim 1, in which the hydrogenation catalyst contains cobalt in an amount, expressed as oxide, between 0.5 wt. % and 15 wt. % with respect to the weight of the catalyst.

6. Process according to claim 1, in which the hydrogenation catalyst also contains molybdenum in an amount, expressed as oxide, between 1 wt. % and 20 wt. % with respect to the weight of the catalyst.

7. Process according to claim 1, in which the hydrolysis catalyst also contains at least 1 wt. %, preferably between 0.5 wt. % and 10 wt. %, of at least one sulphate or silicate of an alkali metal or alkaline earth metal or of a rare earth.

8. Process according to claim 1, in which the hydrolysis catalyst also contains a metal chosen from nickel, cobalt, molybdenum and tungsten.

9. Process according to claim 8, in which the catalyst comprises:
titanium oxide or alumina in an amount between 60 wt. % and 99.8 wt. % with respect to the weight of the catalyst,
at least one metal chosen from nickel, cobalt, molybdenum and tungsten in an amount, expressed as oxide, between 0.2 wt. % and 40 wt. %, preferably between 1 wt. % to 25 wt. % with respect to the weight of the catalyst.

10. Process according to claim 1, in which the stages a) and b) are carried out in the same reactor comprising a hydrogenation catalyst bed (13) and a hydrolysis catalyst bed (14), the beds (13, 14) being arranged with respect to one another in the reactor so that the gas to be treated comes in to contact with the hydrogenation catalyst bed (13) before the hydrolysis catalyst bed (14).

11. Process according to claim 1, in which stage a) is carried out at a pressure between 0.1 and 5 MPa and a HSV between 1000 and 4000 h⁻¹.

12. Process according to claim 1, in which stage b) is carried out at a pressure between 0.1 and 5 MPa and a HSV between 1000 and 4000 h⁻¹.

13. Process according to claim 1, comprising a stage c) in which the effluent rich in H₂S leaving the stage b) is treated in a unit for trapping H₂S or for converting H₂S into elementary sulphur.

14. Process according to claim 1, in which a liquid/gas separation of the gas to be treated is implemented before carrying out the hydrogenation stage a).

15. Process according to claim 1, in which the gas to be treated is obtained from coal or petcoke or even biomass gasification units or from calcination furnaces of carbon black production units.
